(19) United States
(12) Patent Application Publication
(10) Pub. No.: US 2001/0044625 A1
(43) Pub. Date: Nov. 22, 2001

(54) Catheter for circular tissue ablation and methods thereof

(76) Inventors: Cary Hata, Tustin, CA (US); Weng-Kwen Raymond Chia, Irvine, CA (US)

Correspondence Address: Cary Hata, 2500 San Simon St., Tustin, CA, 92782, US

(21) Appl. No.: 09/874,632

(22) Filed: Jun. 4, 2001

Related U.S. Application Data
(63) Continuation-in-part of application No. 09/351,080, filed on Jul. 9, 1999, now Pat. No. 6,241,727.
Application No. 09/351,080 is a continuation-in-part of application No. 09/085,543, filed on May 27, 1998, now Pat. No. 6,238,390.

(51) Int. Cl. A61B 18/14

(52) U.S. Cl. 606/41

(57) ABSTRACT

This invention discloses an improved catheter system having a movable electrode on a deployable wire loop assembly for ablating at least one focal point of a circumferential region of tissue at a location where a pulmonary vein extends from an atrium in a patient, and a method comprising providing radiofrequency energy to the movable electrode for contacting and ablating said at least one focal point.

### CROSS-REFERENCE TO RELATED APPLICATIONS

This application is a continuation-in-part of U.S. patent application Ser. No. 09/351,080 filed on Jul. 9, 1999, now U.S. Pat. No. 6,241,727 which is a continuation-in-part of U.S. patent application Ser. No. 09/085,543, filed on May 27, 1998, now U.S. Pat. No. 6,238,390.

### FIELD OF THE INVENTION

The present invention generally relates to improved constructions for a catheter system and methods thereof More particularly, this invention relates to a catheter system and methods for ablating tissues via a steerable ablation catheter comprising a movable electrode for focal circular ablation in a pulmonary vein as a therapy for atrial ablation management.

### BACKGROUND OF THE INVENTION

The heart includes a number of normal pathways, referring as “electrical conductivity pathways” in this invention, that are responsible for the propagation of electrical signals from the upper chamber to the lower chamber necessary for performing normal systole and diastole function. The presence of an arrhythmogenic site or accessory pathway can bypass or short circuit the normal pathway, potentially resulting in very rapid heart contractions, referred to herein as tachycardias.

A variety of approaches, including drugs, implantable pacemakers/defibrillators, surgery, and catheter ablation have been proposed to treat tachycardias. While drugs may be the treatment of choice for many patients, they only mask the symptoms and do not cure the underlying causes. Implantable devices only correct the arrhythmia after it occurs. Surgical and catheter-based treatments, in contrast, will actually cure the problems, usually by ablating the abnormal arrhythmogenic tissue or accessory pathway responsible for the tachycardia. It is important for a physician to accurately steer the catheter tip to the exact site for ablation. Once at the site, it is important for a physician to control the emission of energy to ablate the tissue within or around the heart.

Of particular interest to the present invention are radiofrequency (RF) ablation protocols that have been proven to be highly effective in tachycardia treatment while exposing a patient to minimal side effects and risks. Radiofrequency catheter ablation is generally performed after conducting an initial mapping study where the locations of the abnormal arrhythmogenic site and/or accessory pathway are determined. After a mapping study, an ablation catheter is usually introduced to the target heart chamber and is manipulated so that the ablation tip electrode lies exactly at the target tissue site. Radiofrequency energy or other suitable energy is then applied through the tip electrode to the cardiac tissue in order to ablate the tissue of the arrhythmogenic site or the accessory pathway. By successfully destroying that tissue, the abnormal signal patterns responsible for the tachycardia may be eliminated. However, in the case of atrial fibrillation (AFib) or atrial flutter, multiple arrhythmogenic sites and/or multiple accessory pathways exist. The conventional catheter with a single “stationary” ablation electrode can not effectively cure the symptoms. In the case of paroxysmal atrial fibrillation, a focal circular tissue ablation at about the pulmonary vein is required.

Atrial fibrillation is believed to be the result of the simultaneous occurrence of multiple wavelets of functional re-entry of electrical impulses within the atria, resulting in a condition in which the transmission of electrical activity becomes so disorganized that the atria contracts irregularly. Once considered a benign disorder, AFib now is widely recognized as the cause of significant morbidity and mortality. The most dangerous outcome from AFib is thromboembolism and stroke risk, the latter due to the chaotic contractions of the atria causing blood to pool. This in turn can lead to clot formation and the potential for an embolic stroke. According to data from the American Heart Association, about 75,000 strokes per year are AFib-related.

A catheter utilized in the endocardial RF ablation is inserted into a major vein or artery, usually in the neck or groin area. For paroxysmal AFib indications, a catheter is approached from the atrium to the ostium of a pulmonary vein. The tip section of a catheter is referred to herein as the portion of that catheter shaft containing means for thermal lesion, wherein the tip section may be deflectable and is configured and adapted to form a circular or an irregular-shape loop lesion for focal tissue ablation. The means for focal circular thermal lesion is to position an energy-delivery element adjacent the ostium of the pulmonary vein, whereby the circular ablation means having a firm element, such as a movable electrode, presses against the tissue for focal circular ablation.

The tip section of a conventional electrophysiology catheter that is deflectable usually contains one large “stationary” electrode about 4 to 8 mm in length for ablation purposes. Sometimes, a plurality of long electrodes is used in creating a contiguous linear lesion. However, for creating a circular lesion or a partial circular lesion with uniform lesion quality inside a pulmonary vein, the temperatures at various device-to-tissue contact sites must be as uniform as possible. The potential disadvantages of a large non-movable type electrode in the pulmonary vein ablation procedures may include its difficulty of maneuvering, over-heating, perforating potential, and/or vein wall collapse.

A catheter with a heated balloon has been widely used to apply heat to the balloon-to-tissue sites, for example, U.S. Pat. No. 6,117,101 to Diederich et al. However, the total amount of energy applied from the balloon surface to the tissue sites tends to cause collapse of the pulmonary vein wall due to vascular vasospasm, “energy shock stenosis”, or other mechanisms. The energy shock stenosis is a body's defense against a sudden environmental change, such as heat, cool or injury. Further, the ultrasonic energy of a transducer-induced heated balloon penetrates deep into the tissue of the vein wall that aggravates the vein wall stenosis/collapse problems.

Avitall in the U.S. Pat. No. 5,242,441 teaches a rotatable tip electrode. Said electrode is secured to a high torque wire for rotation and electrical current transmission. The tissue contact site is always the same spot even the electrode is rotated. Moreover, a movable band electrode has been recently introduced to the market to simulate the “rollable electrode” concept. Since the band electrode does not roll, the contact surface spot of the band electrode with tissues is always at the same spot. The potential coagulum at the contact electrode surface spot due to impedance and temperature rises would not go away because of its relatively stationary position of the rotatable tip electrode or the movable band electrode. A multiple electrode tip section of an ablation catheter is difficult to configure and deploy as a circular element for creating a circular tissue lesion inside the pulmonary vein.

U.S. Pat. No. 5,840,076 discloses a balloon type electrode catheter by using a balloon as a medium to create a circular lesion, wherein the balloon is made of a porous material. Said patent discloses a RF circuit by including a patient in the circuit loop, whereby the heat generated by the unipolar RF current at the tissue contact site may unexpectedly hurt the patient. The local fixed heat source of the unipolar means may make the temperature of the fluid inside and around the heated balloon non-uniform.

Diederich et al. in U.S. Pat. No. 6,117,101 discloses a circumferential ablation device assembly, the entire contents of which are incorporated herein by reference. The assembly includes a circumferential ablation element which is adapted to ablate a circumferential region of tissue along a pulmonary vein wall which circumscribes the pulmonary vein lumen, thereby transecting the electrical conductivity of the pulmonary vein against conduction along its longitudinal axis and into the left atrium. Recent research reveals that there is only very few electrical conductivity pathways along the longitudinal axis of the pulmonary vein wall that need ablation. From clinical efficacy standpoints, only a limited heat is required to transect an electrical conductivity pathway; no need to heat the whole circumference of tissue of a pulmonary vein. Excess heat by the Diederich et al. assembly is thought to be the major culprit of the treatment failure, such as vessel collapse or stenosis.

While a radiofrequency electrophysiology ablation procedure using an existing catheter has had promising results, it is critical that the temperature at the catheter tip electrode should be focally uniform and adequate so that at least a focal point of a circumference region of tissue can be ablated for the paroxysmal AFib treatment in a pulmonary vein. Therefore there is a clinical need for an improved catheter and methods for making a partial focal tissue circular ablation in the pulmonary vein employing a movable electrode effective to provide just sufficient RF energy for focal circular ablation in a pulmonary vein as an effective therapy for atrial fibrillation management.

### SUMMARY OF THE INVENTION

In general, it is an object of the present invention to provide a movable electrode to a medical catheter for selective focal treatment. The “movable electrode” is defined in this invention as the electrode that is slidable or rollable along a wire loop assembly for focal tissue ablation, particularly in a pulmonary vein. A catheter system with a movable electrode means has been disclosed in U.S. Pat. No. 5,843,152, U.S. Pat. No. 5,893,884, U.S. Pat. No. 6,238,390, and U.S. Pat. 6,241,727, entire contents of which are incorporated herein by reference.

It is another object of the present invention to provide a method for ablating at least one focal point of a circumferential region of tissue at a location where a pulmonary vein extends from an atrium in a patient, the method comprising providing radiofrequency energy to a movable electrode for contacting and ablating the at least one focal point. The at least one focal point comprises a passing point of an electrical conductivity pathway, wherein radiofrequency energy is provided sufficient to transect the electrical conductivity pathway at around that passing point.

It is a further object of the present invention to provide a method comprising ablating a second of the at least one focal point by positioning the movable electrode to the second focal point.

In one embodiment, a medical catheter for ablating at least one focal point of a circumferential region of tissue at a location where a pulmonary vein extends from an atrium in a patient comprises a flexible catheter body having a distal section, a distal end, a proximal end, and at least one lumen extending therebetween, the distal end having an opening. The catheter further comprises a control element defining a distal portion operably within the distal section of the catheter body and a proximal portion associated with, and extending along, one of the at least one lumen of the catheter body to an area adjacent the proximal end of the catheter body.

In a further embodiment, the catheter comprises a movable electrode mounted on a deployable wire loop assembly, wherein a first end of the wire loop assembly is securably coupled to the distal portion of the control element configured for deploying the wire loop assembly out of the opening of the distal end of the catheter body; and a control mechanism, associated with the handle and the control element, configured to secure the proximal portion of the control element in predetermined relation to the catheter body and adapted for deploying the wire loop assembly out of the opening. In an alternate embodiment, the wire loop assembly may be preshaped and configured to form a loop upon deployment at an angle relative to an axis of the catheter body.

In a preferred embodiment, the catheter body defines a size and flexibility suitable for percutaneous insertion into a human body. The medical catheter may further comprises a second end of the wire loop assembly to be positioned within one of the at least one lumen of the catheter body. The movable electrode is controlled to move forward and backward by a control wire attached to the movable electrode, and a radiofrequency source is coupled to the movable electrode for ablating the at least one focal point of a circumferential region of tissue of the pulmonary vein.

The medical catheter of the present invention may further comprise an expandable element at the distal section of the medical catheter adapted for stabilizing the distal section at about the circumferential region of tissue of the pulmonary vein for target tissue ablation. The expandable element of the medical catheter of the present invention may be a basket assembly having a plurality of expandable members or an inflatable balloon.

The inflatable balloon may be made of a material selected from the group consisting of silicone, polyurethane, polyethylene, cross-linked polyethylene, conductive silicone, polyethylene terephthalate, latex, semi-permeable membrane, and nylon. And a sodium chloride containing liquid may be used to inflate the inflatable balloon. The concentration of sodium chloride is preferably around the physiology compatible range.

The medical catheter of the present invention may further comprise a steering mechanism at the handle for controlling deflection of the catheter distal section. Usually a rotating ring or a push-pull plunger is employed in the steering mechanism. In another embodiment, the steerable medical catheter comprises a bi-directional deflection or multiple curve deflection of the tip section. One end of the steering wire is usually attached at certain point of the distal section of the catheter body. The other end is attached to the steering mechanism at the handle. The steering mechanism on a steerable catheter or device is well known to those who are skilled in the art.

The medical catheter may further comprise a radiofrequency source or a RF energy generator, wherein RF energy is delivered to the movable electrode through an electrical conductor for ablating at least one focal point of a circumferential region of tissue of a pulmonary vein. The electrode may be made of a material selected from the group consisting of platinum, iridium, gold, silver, stainless steel, and Nitinol.

In a particular embodiment, one end of the electrical conductor is coupled to the movable electrode while the other end is secured to a contact pin of the connector secured at the proximal end of the handle. Therefrom, the electrical conductor is connected to an EKG monitor for recording and displaying of the endocardial, epicardial, or endoluminal electrical signal from the electrode.

The catheter for circular tissue ablation and methods thereof of the present invention has several significant advantages over known catheters or ablation techniques. In particular, a movable electrode inside a pulmonary vein is for ablating at least one focal point of a circumferential region of tissue at a location where a pulmonary vein extends from an atrium in a patient, and the improved methods of this invention may result in a focal circular tissue ablation that is highly desirable in paroxysmal atrial fibrillation treatments.

### BRIEF DESCRIPTION OF THE DRAWINGS

Additional objects and features of the present invention will become more apparent and the invention itself will be best understood from the following Detailed Description of the Exemplary Embodiments, when read with reference to the accompanying drawings.

FIG. 1 is an overall view of a medical catheter having a movable electrode on a deployable wire loop assembly and an optional expandable element at a distal section of the catheter constructed in accordance with the principles of the present invention.

FIG. 2 is a close-up side view of the distal section of the medical catheter comprising a movable electrode on a deployable wire loop assembly and an optional inflatable balloon as a stabilizer for the medical catheter.

FIG. 3 is a front cross-sectional view, section 1-1 of FIG. 2, of the wire loop assembly along with a movable electrode.

FIG. 4 is a perspective view, section 2-2 of FIG. 3, of the movable electrode on a wire loop assembly of the medical catheter.

FIG. 5 is a transverse cross-sectional view, section 2-2 of FIG. 3, of the attachment setup of a movable electrode on a wire loop assembly.

FIG. 6 is a simulated view of the catheter of the present invention in contact with the tissue of a pulmonary vein.

### DETAILED DESCRIPTION OF THE EXEMPLARY EMBODIMENTS

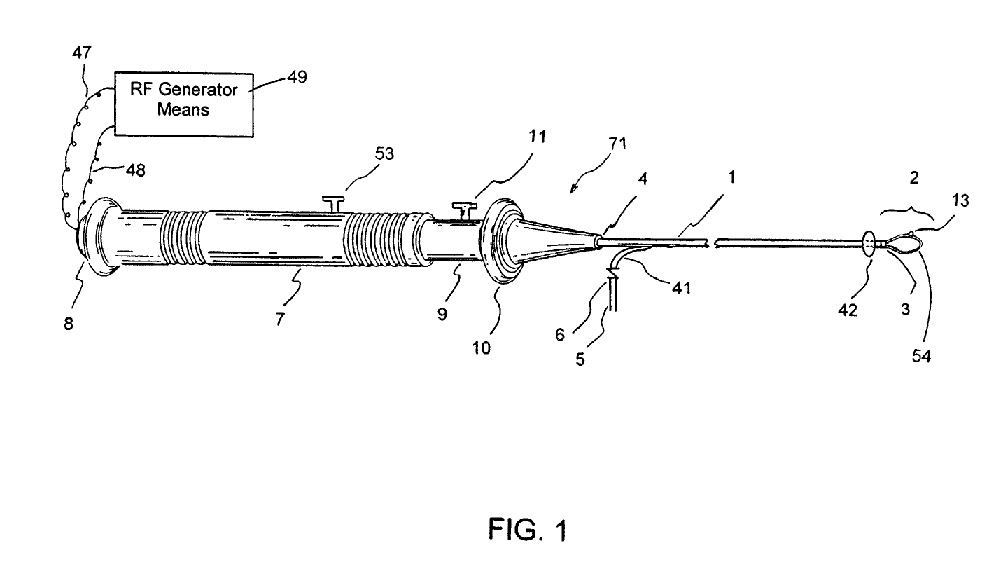

What is shown in FIG. 1 to FIG. 6 is an embodiment of a medical catheter for ablating at least one focal point of a circumferential region of tissue at a location where a pulmonary vein extends from an atrium in a patient.

FIG. 1 shows an overall view of the medical catheter having a movable electrode on a deployable wire loop assembly and an optional expandable element at a distal section of the catheter constructed in accordance with the principles of the present invention. A medical catheter 71 constructed in accordance with the principles of the present invention comprises a flexible catheter body 1 having a distal section 2, a distal end 3, a proximal end 4, and at least one lumen extending therebetween, wherein the distal end 3 has an opening 51. In an illustrative embodiment for a balloon-type expandable element 42, the catheter 71 has an inflation fluid passageway 41. A handle 7 is attached to the proximal end 4 of the catheter body 1, wherein the handle has a cavity. The catheter body is configured in such a way that it defines a size and flexibility suitable for percutaneous insertion into a human body

A connector 8 secured at the proximal end of the catheter 71, is part of the handle section 7. The handle has one optional steering mechanism 9. The steering mechanism 9 is to deflect the distal section 2 of the catheter body 1 for catheter maneuvering and positioning. By pushing forward the front plunger 10 of the handle 7, the distal section 2 of the catheter body deflects to one direction. By pulling back the front plunger 10, the distal section returns to its neutral position. In another embodiment, the steering mechanism 9 at the handle 7 comprises means for providing a plurality of deflectable curves on the distal section 2 of the catheter body 1.

In a preferred embodiment, the medical catheter system further comprises an expandable element at a distal section of the catheter 71 adapted for stabilizing the distal section 2 at about the circumferential region of the pulmonary vein. The expandable element may comprise an expandable basket with a plurality of expandable members or an inflatable balloon 42. The optional inflatable balloon 42 is mounted at the distal section 2, proximal to the distal end 3. The medical catheter system may further comprise a fluid inflation mechanism 5 close to the proximal end 4 of the catheter shaft 1. A control valve 6 is provided to the fluid inflation mechanism 5 which is externally connected to a fluid supply source having a syringe or pump for inflating the inflatable balloon 42. The inflation passageway 41 is connected to a lumen that opens into and is in communication with an interior 43 of the inflatable balloon 42.

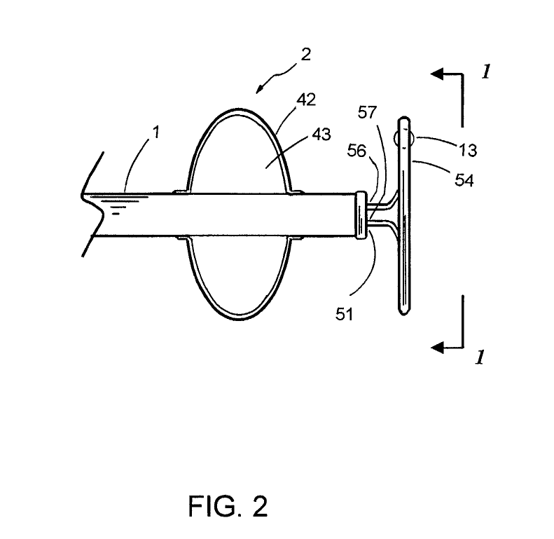

FIG. 2 shows a close-up side view of the distal section 2 of the medical catheter 71 comprising a movable electrode 13 on a wire loop assembly 54 and an optional inflatable balloon 42 as a stabilizer for the medical catheter. In one embodiment, the wire loop assembly 54 is preshaped and configured to form a loop upon deployment at an angle relative to an axis of the catheter body 1. In other words, the plane formed by the loop and the plane formed by the catheter body intersect to each other. The angle may range from a few degrees to more than 90 degrees. The movable electrode is positioned along an exterior circumference of a deployable wire loop assembly of a delivery apparatus, wherein the construction material of the wire loop assembly is non-conductive or insulative from radiofrequency current. The delivery apparatus may be a medical catheter configured to be inserted into the pulmonary vein by a percutaneous procedure or the delivery apparatus may be a medical wire assembly configured to be inserted into the pulmonary vein by a percutaneous procedure.

The medical catheter of the present invention comprises a control element defining a distal portion operably within the distal section 2 of the catheter body 1. The control element also comprises a proximal portion associated with, and extending along, one of the at least one lumen of the catheter body 1 to an area adjacent the proximal end 4 of the catheter body. The control element can be a wire, a flat wire, a cylinder, a rod, any flexible material and construction that has the strength and pushability, and the like.

A control mechanism 11, associated with the handle 7 and the control element is configured to secure the proximal portion of the control element in predetermined relation to the catheter body. By operating the control mechanism 11 on the handle 7, the wire loop assembly 54 may be operated forward or backward so that the wire loop assembly is deployable out of the opening 51 of the catheter body 1.

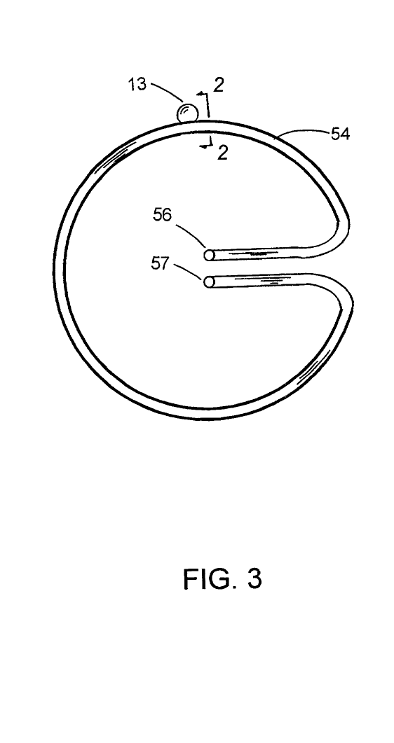

FIG. 3 shows a front cross-sectional view, section 1-1 of FIG. 2, of the wire loop assembly 54 along with a movable electrode 13. The movable electrode 13 is mounted on the wire loop assembly 54, wherein a first end 56 of the wire loop assembly 54 is coupled to the distal portion of the control element. The second end 57 of the wire loop assembly 54 may be positioned within one of the at least one lumen of the catheter body and secured at certain point along the catheter body 1.

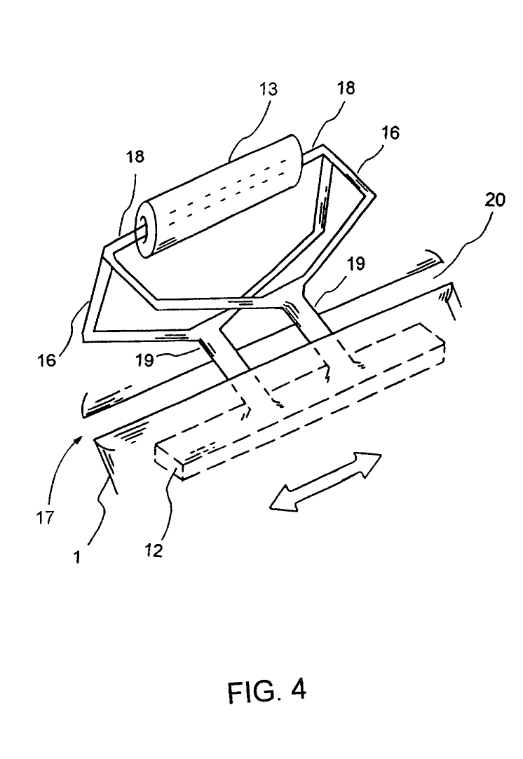

FIG. 4 shows a perspective view, section 2-2 of FIG. 3, of the movable electrode 13 on the wire loop assembly 54 of the medical catheter. In one exemplary embodiment, the electrode assembly comprises a movable electrode 13, a support 16 and an anchoring leg means 19 disposed inside an elongated open groove 17 for the movable electrode 13, wherein the movable electrode is positioned on the support and wherein the support is connected to the anchoring leg means which is then secured onto a control wire 12 of the electrode deployment mechanism 53. The movable electrode is generally controlled to move forward and backward riding on the wire loop assembly 54 by a control wire attached to the movable electrode 13, wherein the other end of the control wire is secured to the electrode deployment mechanism 53 on the handle 7.

The electrode may be selected from a group consisting of a cylindrical roller, a ball-type roller, an oval-type roller, a porous roller, a roller with studded surface and the like. The electrode is preferably made of conductive material, while the surfaces of the shafts 18, supports 16, the anchoring leg means 19, and the control wire 12 are preferably covered with an insulating material or insulated. The anchoring leg means 19 is secured to the control wire 12 through an open slit 20 of the open groove 17, wherein the control wire 12 is preferred to be made of a flat wire. When the control wire is pushed forward by the electrode deployment mechanism 53, the movable electrode 13 moves forward, too. The movable electrode 13 tends to roll or move forward even when it contacts the tissues.

In an illustrative example for radiofrequency ablation principles, the electrode 13 has an insulated electrical conducting wire (not shown) secured to the electrode, wherein the wire passes through one of the at least one lumen of the catheter body 1 and is secured to a contact pin of the connector 8 at the proximal end of the handle 7. The returning conducting wire 47 or 48 from the end of the connector is externally connected to a RF generator means 49 during an electrophysiology ablation procedure. The radiofrequency ablation can be operated either as a unipolar mode or a bipolar mode.

A temperature sensor, either a thermocouple means or a thermister means, may be constructed at adjacent the electrode 13 to measure the tissue temperature when RF energy is delivered. The temperature sensing wire (not shown) from the thermocouple or thermister is connected to one of the contact pins (not shown) of the connector 8 and externally connected to a temperature controller. The temperature reading is thereafter relayed to a closed-loop control mechanism to adjust the RF energy output. The RF energy delivered is thus controlled by the temperature sensor reading or by a pre-programmed control algorithm. The ablation catheter system further comprises a steering mechanism 9 at the handle 7 for controlling deflection of the distal section 2. Usually a rotating ring or a push-pull plunger is employed in the steering mechanism.

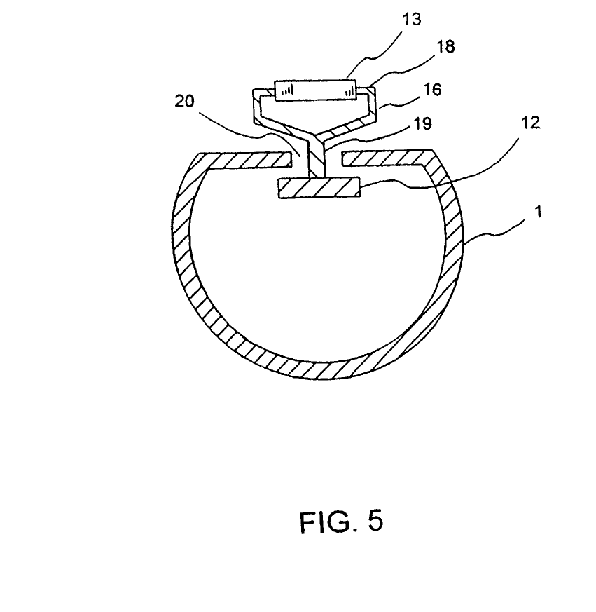

In a preferred embodiment, FIG. 5 shows a transverse cross-sectional view, section 2-2 of FIG. 3, of the attachment setup of a movable electrode on a wire loop assembly. The electrode assembly comprises a movable electrode 13, electrode shafts 18, supports 16, and anchoring leg means 19. The anchoring leg means 19 is firmly secured on the forward control wire 12, which is controlled by the electrode deployment mechanism 53 for moving the electrode 13 forward or backward for focal ablation.

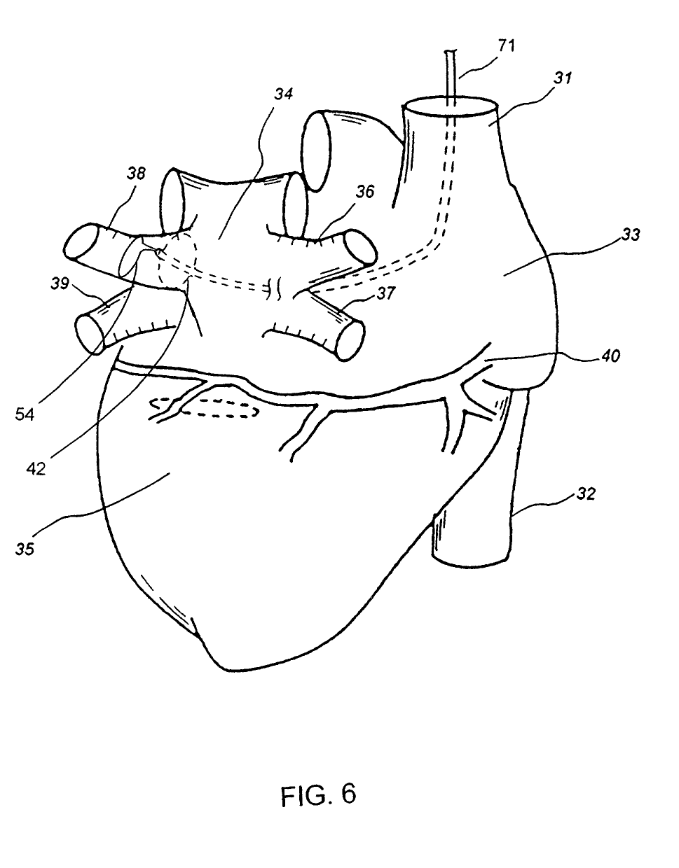

FIG. 6 shows a simulated view of the medical catheter of the present invention in contact with the tissue of a pulmonary vein for paroxysmal atrial fibrillation treatment. To better illustrate the application of the present invention, a human heart is shown in FIG. 6. Blood returning from superior vena cava 31 or inferior vena cava 32 flows back to the right atrium 33. A coronary sinus 40 is part of the coronary artery system to provide nutrient to the epicardial heart tissue, wherein the heart also comprises a left atrium 34, a left ventricle 35 and a right ventricle. A medical catheter 71 of the present invention passes through the superior vena cava 31 into the right atrium 33. The catheter with a delivery sheath or a guiding catheter passes through the septum into the left atrium 34 for paroxysmal AFib treatment by using a standard trans-septal procedure. A normal person has four pulmonary veins: right superior pulmonary vein 36, right inferior pulmonary vein 37, left superior pulmonary vein 38, and left inferior pulmonary vein 39.

In one example, a catheter 71 is inserted into the left atrium while its distal tip section is inserted into the left superior pulmonary vein 38. After the distal section of the catheter 71 is inside the vein 38, the expandable element, for example an inflatable balloon 42, is deployably adapted for stabilizing the distal section at about the circumferential region of the pulmonary vein. Then the wire loop assembly 54 is deployed. In a preferred embodiment, the deployed wire loop assembly is about perpendicular to the catheter body. Radiofrequency energy is applied to the movable electrode once an arrhythmogenic site for treatment is identified.

A method for operating a catheter system of the present invention comprises percutaneously introducing the catheter system through a blood vessel to the pulmonary vein of the heart; positioning the distal section inside the pulmonary vein; deploying the wire loop assembly; positioning the movable electrode at the target lesion site; and applying RF energy to the electrode for tissue ablation, wherein RF energy is provided from an external RF energy generator means to the electrode through an electrical conductor.

In general, a method for ablating at least one focal point of a circumferential region of tissue at a location where a pulmonary vein extends from an atrium in a patient, the method comprising providing radiofrequency energy to a movable electrode for contacting and ablating said at least one focal point. The method may further comprise ablating a second of said at least one focal point by positioning said movable electrode to said second focal point.

From the foregoing, it should now be appreciated that an improved medical catheter system having a movable electrode on a deployable wire loop assembly for ablating at least one focal point of a circumferential region of tissue at a location where a pulmonary vein extends from an atrium in a patient has been disclosed. While the invention has been described with reference to a specific embodiment, the description is illustrative of the invention and is not to be construed as limiting the invention. Various modifications and applications may occur to those skilled in the art without departing from the true spirit and scope of the invention as described by the appended claims.

What is claimed is:

1. A method for ablating at least one focal point of a circumferential region of tissue at a location where a pulmonary vein extends from an atrium in a patient, the method comprising providing radiofrequency energy to a movable electrode for contacting and ablating said at least one focal point.

2. The method according to claim 1, wherein said at least one focal point comprises a passing point of an electrical conductivity pathway.

3. The method according to claim 2, wherein radiofrequency energy is provided sufficient to transect said electrical conductivity pathway.

4. The method according to claim 1, the method further comprising ablating a second of said at least one focal point by positioning said movable electrode to said second focal point.

5. The method according to claim 1, wherein said movable electrode is positioned along an exterior circumference of a deployable wire loop assembly of a delivery apparatus, construction material of said wire loop assembly being nonconductive or insulative from radiofrequency current.

6. The method according to claim 5, wherein the delivery apparatus is a medical catheter configured to be inserted into the pulmonary vein by a percutaneous procedure.

7. The method according to claim 6, wherein the medical catheter further comprises an expandable element at a distal section of said catheter adapted for stabilizing said distal section at about the circumferential region of the pulmonary vein.

8. The method according to claim 7, wherein the expandable element is an inflatable balloon.

9. The method according to claim 7, wherein the expandable element is a basket assembly having a plurality of expandable members.

10. The method according to claim 5, wherein the delivery apparatus is a medical wire assembly configured to be inserted into the pulmonary vein by a percutaneous procedure.

11. A medical catheter for ablating at least one focal point of a circumferential region of tissue at a location where a pulmonary vein extends from an atrium in a patient, the medical catheter comprising:
a flexible catheter body having a distal section, a distal end, a proximal end, and at least one lumen extending therebetween, the distal end having an opening;
a handle attached to the proximal end of the catheter body, wherein the handle has a cavity;
a control element defining a distal portion operably within the distal section of the catheter body and a proximal portion associated with, and extending along, one of the at least one lumen of the catheter body to an area adjacent the proximal end of the catheter body;
a movable electrode mounted on a deployable wire loop assembly, wherein a first end of said wire loop assembly is coupled to said distal portion of the control element configured for deploying said wire loop assembly out of the opening of the distal end of the catheter body; and
a control mechanism, associated with the handle and the control element, configured to secure the proximal portion of the control element in predetermined relation to the catheter body and adapted for deploying said wire loop assembly.

12. The medical catheter according to claim 11, wherein the catheter body defines a size and flexibility suitable for percutaneous insertion into a human body.

13. The medical catheter according to claim 11, wherein a second end of said wire loop assembly is positioned within one of the at least one lumen of said catheter body.

14. The medical catheter according to claim 11, wherein the movable electrode is controlled to move forward and backward by a control wire attached to said movable electrode.

15. The medical catheter according to claim 14 further comprising an expandable element at the distal section of said medical catheter adapted for stabilizing said distal section at about the circumferential region of tissue of the pulmonary vein.

16. The medical catheter according to claim 11, wherein the wire loop assembly is preshaped and configured to form a loop upon deployment at an angle relative to an axis of the catheter body.

17. The medical catheter according to claim 15, wherein the expandable element is a basket assembly having a plurality of expandable members.

18. The medical catheter according to claim 15, wherein the expandable element is an inflatable balloon.

19. The medical catheter according to claim 11, wherein a radiofrequency source is coupled to the movable electrode for ablating at least one focal point of a circumferential region of tissue of said pulmonary vein.

20. A method for ablating at least one focal point of a circumferential region of tissue at a location where a pulmonary vein extends from an atrium in a patient, the method comprising providing radiofrequency energy to a movable electrode of a medical catheter for contacting said at least one focal point, the medical catheter comprising:
a flexible catheter body having a distal section, a distal end, a proximal end, and at least one lumen extending therebetween, the distal end having an opening;
a handle attached to the proximal end of the catheter body, wherein the handle has a cavity;
a control element defining a distal portion operably within the distal section of the catheter body and a proximal portion associated with, and extending along, one of the at least one lumen of the catheter body to an area adjacent the proximal end of the catheter body;
a movable electrode mounted on a deployable wire loop assembly, wherein a first end of said wire loop assembly is coupled to said distal portion of the control element configured for deploying said wire loop assembly from the distal section of the catheter body; and
a control mechanism, associated with the handle and the control element, configured to secure the proximal portion of the control element in predetermined relation to the catheter body and adapted for deploying said wire loop assembly out of the opening of the catheter body.
